(19) United States
(12) Patent Application Publication
(10) Pub. No.: US 2020/0022933 A1
(43) Pub. Date: Jan. 23, 2020

(54) SURFACTANTS AND COMPOSITIONS FOR TREATMENT OF VIRAL SKIN CONDITIONS

(71) Applicant: Mehmet Nevzat Pisak, Istanbul (TR)

(72) Inventor: Mehmet Nevzat Pisak, Istanbul (TR)

(21) Appl. No.: 16/585,959

(22) Filed: Sep. 27, 2019

Related U.S. Application Data
(62) Division of application No. 15/547,731, filed on Jul. 31, 2017.

(51) Int. Cl. A61K 31/185; A61K 47/38; A61K 47/34; A61K 47/32; A61K 9/06; A61P 31/22; A61K 9/00; A61K 9/08

(52) U.S. Cl. CPC A61K 31/185; A61K 47/38; A61K 47/34; A61K 9/08; A61K 9/06; A61P 31/22; A61K 9/0014; A61K 47/32

(57) ABSTRACT

Disclosed is a surfactant, or a pharmaceutically acceptable salt or a stereoisomer thereof, and a composition including the surfactant, for post infection treatment of a double stranded DNA skin virus from the Herpesviridae or Papillomaviridae families. The composition may include an anionic or amphoteric surfactant, or a pharmaceutically acceptable salt or a stereoisomer thereof, wherein the surfactant further includes: a hydrophilic moiety selected from the group consisting of a carbonate, a sulfonate, and a sulfate; a polyol compound and/or a cross-linked polyacrylate polymer, and at least one pharmaceutically acceptable excipient. Methods of using the surfactant and composition are also disclosed.

### TECHNICAL FIELD

This invention relates to topical surfactant formulations for the treatment of viral skin infections caused by double stranded DNA viruses, and in particular viruses from the Herpesviridae and Papillomaviridae families.

### BACKGROUND

Skin infections of viral origin can be caused by DNA and RNA type viruses, both of which are obligatory intracellular parasites. Such skin infections can be quite common leading to manifestations such as lesions, rashes, or warts, and resulting in symptoms ranging from great discomfort to even cancer.

Viral skin infections caused by the Herpesviridae and Papillomaviridae family of viruses, including Herpes simplex type 1 (HSV-1), Herpes simplex type 2 (HSV-2), Varicella-zoster virus (VZV), Epstein-Barr virus (EBV), Human cytomegalovirus (HCV), Human herpesvirus type 8, and Human papillomavirus (HPV), are very common among humans. At least 90% of adults are infected with one of these viruses, and a latent form of the virus remains in most people. In total, there are 8 herpesvirus types that infect humans: HSV-1, HSV-2, VZV, EBV, HCV, human herpesvirus 6, human herpesvirus 7, and Kaposi's sarcoma-associated herpesvirus.

HSV-1 and HSV-2 can cause orolabial herpes and genital herpes. VZV can cause chicken-pox and shingles (herpes zoster). EBV can cause mononucleosis, and HCV can cause cutenous lesions depending upon the immunocompromised state of the host.

The HSV-1 and HSV-2 virus replicates initially in epithelial cells, producing a characteristic vesicle on an erythematous base. It then ascends sensory nerves to the dorsal root ganglia, where, after an initial period of replication, it establishes latency. During reactivated infection, the virus spreads distally from the ganglion and manifests as new cutaneous and/or mucosal lesions.

VZV is usually transmitted by droplets and replicates initially in the nasopharynx. In seronegative individuals, viremia and chickenpox ensue. Latency is established in dorsal root ganglia, and virus reactivation results in virlon transport down sensory nerves. Reactivation of latent virus (usually in adults) causes herpes zoster (shingles), manifesting as a vesicular rash with a dermatomal distribution and acute neuritis.

HPV is a double stranded DNA virus from the Papillomaviridae family, which induces infections In mucous membranes and/or skin keratinocytes. They are also very common. Skin conditions caused by a human papillomavirus infection include, but are not limited to, warts, lesions, and epidermodysplasia verruciformis. Although warts are considered as a benign infection, they can sometimes be contagious or cause great discomfort depending on their appearance and location. Trauma and maceration facilitate initial epidermal inoculation of human papillomavirus. Local and systemic immune factors appear to influence spread of the infection, and immunosuppressed patients are at particular risk of developing generalized lesions that are difficult to treat. Many warts regress spontaneously after a few months but others can persist for years and recur at the same or different sites even with treatment. Genital warts (condyloma acuminata and condyloma acuminatum) are the primary cause of cervical cancers.

Thus, the complexity and opportunistic nature of viral pathogens that cause viral skin disease makes it very difficult for a person's adaptive and innate immune system to eradicate the viral pathogen on its own. Anti-viral drugs which are mainly DNA synthesis inhibitors, such as nucleoside, purine and prymidine analogues (acyclovir, valacyclovir, penciclovir, famciclovir, trifluridine, foscarnet, etc.), can integrate themselves into the DNA of replicating infected cells and can act in a mutagenic manner. Furthermore viral pathogens tend to mutate over time and are prone to drug resistance.

Although there are conventional treatments for viral skin diseases, these have either been inadequate or caused serious side effects, such as affecting healthy cells in the skin or in other organs. Common side effects include nausea and diarrhea. Potentially serious side effects include kidney problems and low platelets.

An alternative therapy has been the use of immunomodulatory agents to inhibit the viral synthesis in infected cells. Immunomodulatory agents include the TLR7 agonist immiquimod and interferon alpha. The side effects associated with these treatments can also be severe and they are not considered as an adequate treatment option in certain cases. The side effects stated above make it difficult to directly target the virus causing the viral skin disease, without harming healthy cells.

Surfactants have previously been disclosed as virucides and microbicides to be used as preventive measures prior to infection. Surfactants have been previously disclosed as disinfecting agents and as a prophylactic measure against sexually transmitted diseases. Prior studies and publications have also used formulations of surfactants as prophylactic agents meant for the prevention of the virus infection by inactivation of the virus, prior to the inoculum of the host cells, and prior to their manifestations on the skin as lesions, papillomas, and the like. Thus, prior research has been directed towards protection and prevention rather than the treatment of skin conditions after the infection has occurred.

However, there remains a need for safe, efficient, and effective treatment of skin conditions in patients infected with double stranded DNA viruses, and in particular skin conditions caused by HSV-1, HSV-2, VZV, and HPV.

### SUMMARY

Compounds, compositions, and methods of the present invention advantageously and surprisingly allow for the treatment of DNA type viral skin infections and their manifestations after occurrence of infection in a safe manner with minimal side effects.

It has been unexpectedly discovered that a topical formulation of surfactants, such as anionic surfactants, of the present invention has unparalleled in-vivo therapeutic activity in humans and can be safely used for the treatment of viral skin infections caused by double stranded DNA viruses, post inoculum or post infection in humans, with superior safety and efficacy compared to current treatments.

In particular, within the subgroup of DNA type viruses, the present invention may be used for the treatment of viral skin infections and conditions caused by double stranded DNA viruses, especially viruses from the Herpesviridae and Papillomaviridae families. For example, the present invention provides for topical application of a surfactant composition for the treatment of viral skin infections and conditions caused by alphaherpesviruses, including HSV-1, HSV-2, and VZV from the Herpesviridae family and HPV from the Papillomaviridae family.

In one embodiment, a surfactant, or a pharmaceutically acceptable salt or a stereoisomer thereof, is provided for post infection treatment of a double stranded DNA skin virus from the Herpesviridae or Papillomaviridae families.

The above surfactant may have the following alternative components or elements, which may also be combined in various applicable and functioning combinations within the scope of the present invention.

The surfactant may have at least one of the following formulas (I) to (XXIII):

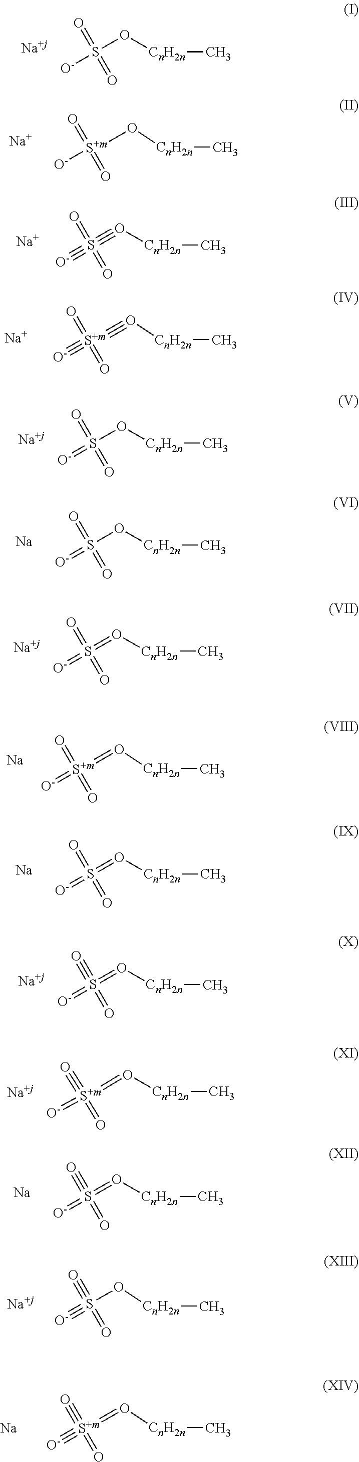

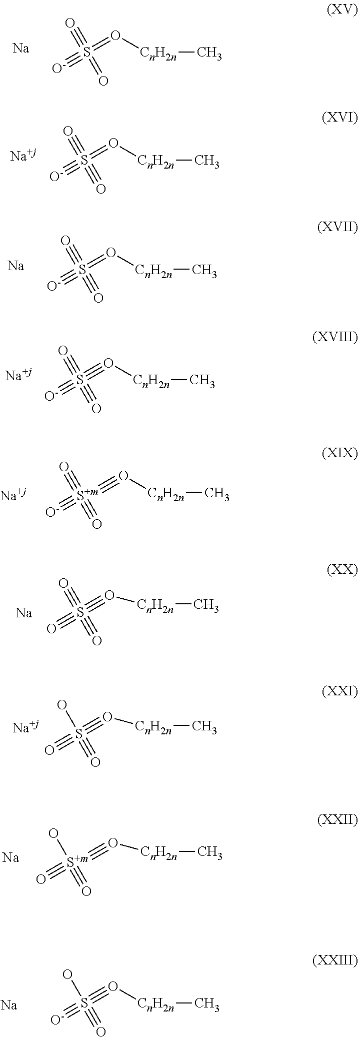

wherein
m is an integer from 1 to 10;
n is an integer from 3 to 31; and
j is an integer from 1 to 10.

In the surfactant formulas above, n may also be an integer from 5 to 21, 7 to 15, or 7 to 25 in other embodiments. The surfactant may be selected from the group consisting of an anionic surfactant, an amphoteric surfactant, and mixtures thereof. The surfactant may further comprise a hydrophilic moiety selected from the group consisting of a carbonate, a sulfonate, and a sulfate.

The double stranded DNA virus from the Herpesviridae or Papillomaviridae families treatable by the surfactant may include the human papilloma virus, herpes simplex virus type 1, herpes simplex virus type-2, and the herpes zoster disease.

In another embodiment, a composition for post infection treatment of a double stranded DNA virus from the Herpesviridae or Papillomaviridae families is provided, the composition comprising: an anionic or amphoteric surfactant, or a pharmaceutically acceptable salt or a stereoisomer thereof, wherein the surfactant further comprises a hydrophilic moiety selected from the group consisting of a carbonate, a sulfonate, and a sulfate; a polyol compound and/or a cross-linked polyacrylate polymer; and at least one pharmaceutically acceptable excipient.

The above composition may have the following alternative components, which may also be combined in various applicable and functioning combinations within the scope of the present invention.

The anionic or amphoteric surfactant of the composition may have one of the formulas (I)-(XXIII) as listed above. The surfactant may be selected from the group consisting of sodium lauryl sulphate, sodium dodecyl sulphate, potassium dodecyl sulphonate, sodium dodecyl benzene sulphonate, sodium salt of lauryl polyoxyethylene sulphate, lauryl polyethylene oxide sulfonate, dioctyl ester of sodium sulphosuccinic acid or sodium lauryl sulphonate, ammonium lauryl sulfate, sodium 2-ethylhexyl sulfate, sodium octyl sulfate, lithium lauryl sulfate and their salts, and a combination thereof.

The polyol compound may be selected from the group consisting of ethylene glycol, propylene glycol, butylene glycol, hexylene glycol, propylene glycol monocaprylate, and a combination thereof.

The cross-linked polyacrylate polymer may be selected from the group consisting of polyacrylates, carbomers, polyvinyl acetates, polymethylmethacrylates, hyaluronic acids, polycarboxylated polysaccharides, carboxyalkyl-polysaccharides, celluloses, dextrans, and a combination thereof.

The surfactant may have one of the following weight percentages in the composition: between 0.1% to 20% by weight of the composition; between 0.3% to 10% by weight of the composition; and between 0.3% to 5% by weight of the composition.

The composition may be formulated for topical administration. The composition may be in the form of a gel, a cream, an ointment, a liquid, a suspension, a solution, an emulsion, a foam, a patch, or an aerosol.

The double stranded DNA virus from the Herpesviridae or Papillomaviridae families treatable by the composition may include the human papilloma virus, herpes simplex virus type 1, herpes simplex virus type-2, and the herpes zoster disease. In one example, the double stranded DNA virus from the Herpesviridae or Papillomaviridae families treatable by the composition is human papilloma virus (HPV) having a manifestation including one or more of common warts, plantar warts, flat warts, anogenital warts, anal dysplasia, epidermodysplasia verruciformis, focal epithelial hyperplasia, oral papillomas, laryngeal papillomatosis, and a combination thereof.

In yet other embodiments, use of the surfactants and compositions as noted above are disclosed. A kit for the treatment of conditions and manifestations of a skin infection such as the human papilloma virus, herpes simplex virus type 1, herpes simplex virus type 2, and the herpes zoster disease, is also disclosed, wherein the kit includes a unit dose of a surfactant and/or composition as described above.

### DETAILED DESCRIPTION

In contrast to past treatment for viral skin infections caused by double stranded DNA viruses from the Herpesviridae and Papillomaviridae families, the present invention discloses a topical treatment comprising a surfactant for the topical treatment of viral skin infections and their manifestations with diminished side effects compared with current treatments.

In some embodiments, the possible compositions and methods of the present invention comprise the topical administration of a surfactant within a suitable carrier, for the treatment of viral skin infections and their manifestations. Advantageously, the present disclosure overcomes the complexity and opportunistic nature of viral pathogens that are the cause of viral skin diseases, which the adaptive and innate immune system finds difficult to eradicate.

Although the present invention is not limited to the theory disclosed herein, it is believed the compounds and the formulations that can be devised with the present invention have a twofold inhibitory effect: it is believed that they bind to the virus, to competitively inhibit virus mediated infection on an intracellular level, and because the lesions produce a large number of virions that infect adjacent tissue, the present invention can also inhibit viral fusion on the manifestations of such lesions, papillomes or vesicular eruptions by inhibition of the viral multiplication cycle. Accordingly, the present invention provides for treatment of the manifestations and conditions on the skin caused by these DNA viral infections.

The adaptive immunity relies on immunological memory but has the distinct disadvantage of time because specific clones need to expand and differentiate into effector cells before they can participate in the host defense. This is why immune responses of adaptive nature are usually delayed for up to 7 days. This delay can be crucial in situations where the host is not able to protect itself against foreign pathogens. The synthesis/fusion of the virus has to be halted for a faster and more efficient immune system recovery.

The new methods and compositions of the present disclosure are based on the findings that individuals infected with a skin virus, such as HPV, HSV-2, and/or herpes zoster, and who show the manifestations and conditions associated with these skin infections, have been, surprisingly, efficiently treated with the topical administration of specific dosages of one or more anionic or amphoteric surfactants, with the surfactant including a polyol compound or a cross-linked polyacrylate polymer or both. The surfactant may also include a hydrophilic moiety, such as a carbonate, sulfonate or sulfate in combination.

In accordance with an embodiment, the compositions and methods of the present invention comprise the administration of an anionic surfactant and a polyol compound and/or a cross-linked polyacrylate polymer within the same composition mixed with other excipients as suitable carriers, for increased efficacy and decreased side effects for the treatment of viral skin diseases and conditions caused by HPV, HSV-1, HSV-2, and/or herpes zoster, and manifestations thereof.

In accordance with another embodiment, the present invention entails compositions and methods comprising the administration of an anionic surfactant and a polyol compound and/or a cross-linked polyacrylate polymer within the same composition for increased efficacy and decreased side effects, in the treatment of skin conditions caused by double stranded DNA viruses, especially viruses from the Herpesviridae and Papillomaviridae families and manifestations thereof.

In accordance with another embodiment, the present invention entails compositions and methods comprising the administration of an anionic surfactant and a polyol compound and/or a cross-linked polyacrylate polymer within the same composition for increased efficacy, increased patient compliance and decreased side effects, in the treatment of the skin conditions caused by HSV-1, HSV-2, VZV, and/or HPV and their manifestations.

As used herein, the term “treating” (or variants thereof) a condition caused by HSV-1, HSV-2, VZV, and/or HPV refers to a pharmaceutical treatment; e.g., the topical administration of one or more agents, such that at least one symptom of the condition is eradicated, diminished or prevented from worsening.

Surfactants may have previously been used in preventive/prophylactic sanitizing formulations as pharmaceutical excipients to increase the absorption of active agents in pharmaceutical compositions or as microbicides. In a surprising and unexpected discovery, an anionic or amphoteric surfactant used in combination with a polyol compound and/or a cross-linked polyacrylate polymer (mixed with suitable excipients known in the art), has rapidly regressed the manifestations of herpes zoster, HSV-1, HSV-2 and several different strains of HPV, such as lesions and papillomes.

Suitable surfactants include, but are not limited to, one or more of an anionic surfactant, an amphoteric surfactant, and mixtures thereof.

In one embodiment, the surfactant may have at least one of the following formulas (I) to (XXIII):

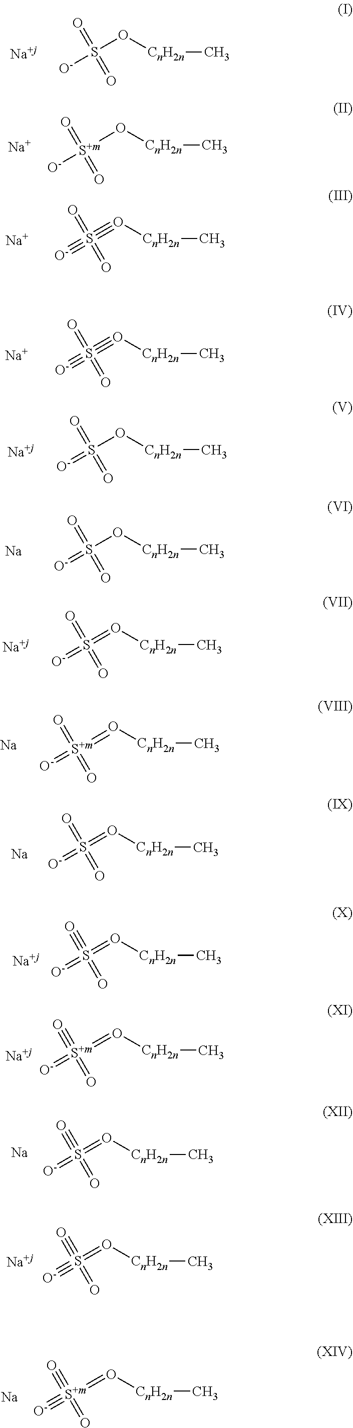

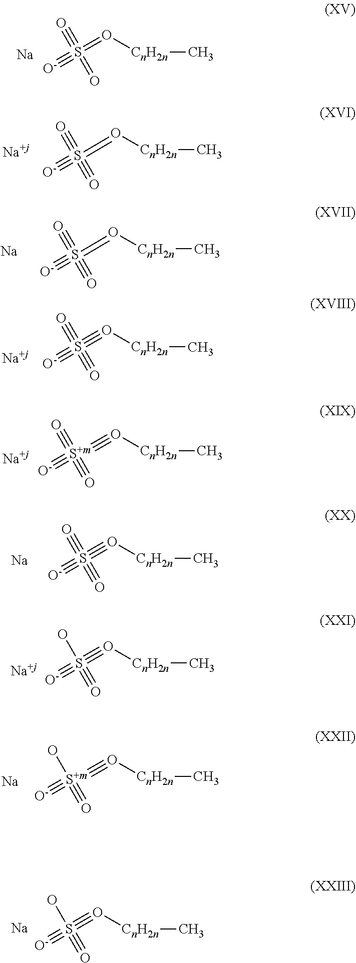

wherein
m is an integer from 1 to 10;
n is an integer from 3 to 31; and
j is an integer from 1 to 10.

The anionic surfactant may include, but is not limited to, one or more of lauryl polyethylene oxide sulfonate, dioctyl ester of sodium sulphosuccinic acid or sodium lauryl sulphonate, ammonium lauryl sulfate, sodium lauryl sulphate, potassium dodecyl sulphonate, sodium dodecyl benzene sulphonate, sodium salt of lauryl polyoxyethylene sulphate sodium 2-ethylhexyl sulfate, sodium octyl sulfate, lithium lauryl sulfate and their salts, and the like.

In one example, an anionic surfactant has about 8 to about 26 carbon atoms, and includes a hydrophilic moiety, such as a carbonate, sulfonate or sulfate.

The amphoteric surfactant may include, but is not limited to, one or more of icinoleamidopropyl betaine, cocamidopropyl betaine, stearyl betaine, stearyl amphocarboxy glycinate, sodium lauraminopropionate, cocoamidopropyl hydroxy sultaine, disodium lauryliminodipropionate, tallowiminodipropionate, cocoampho-carboxy glycinate, cocoimidazoline carboxylate, lauric imidazoline monocarboxylate, lauric imidazoline dicarboxylate, lauric myristic betaine, cocoamidosulfobetaine, alkylamidophospho betaine and the like.

In some embodiments, the anionic surfactant is a compound that may exist in the form of one or more stereoisomers, wherein one or more of those stereoisomers is therapeutically active. In some embodiments, the anionic surfactant comprises a therapeutically active stereoisomer that is substantially free of other stereoisomers. In other embodiments, the anionic surfactant comprises a therapeutically active stereoisomer that has less than about 95%, less than about 75%, less than about 40%, less than about 20%, less than about 10%, less than about 5%, less than about 1%, less than about 0.5%, or less than about 0.1% by weight of other stereoisomers.

Other potential surfactants may include nonionic surfactants and cationic surfactants.

A potential nonionic surfactant may include but is not limited to one or more of polysorbate 80, nonyl phenol polyoxyethylene ether, tridecyl alcohol polyoxyethylene ether, dodecyl mercaptan polyoxyethylene thioether, the lauric ester of polyethylene glycol, the lauric ester of sorbitan polyoxyethylene ether or tertiary alkyl amine oxide, and the like.

A potential cationic surfactant may include but is not limited to one or more of distearyl dimethyl ammonium chloride, stearyl dimethyl benzyl ammonium chloride, stearyl trimethyl ammonium chloride, coco dimethyl benzyl ammonium chloride, dicoco dimethyl ammonium chloride, cetyl pyridinium chloride, cetyl trimethyl ammonium bromide, stearyl amine salts that are soluble in water such as stearyl amine acetate and stearyl amine hydrochloride, stearyl dimethyl amine hydrochloride, distearyl amine hydrochloride, alkyl phenoxyethoxyethyl dimethyl ammonium chloride, decyl pyridinium bromide, pyridinium chloride derivative of the acetyl amino ethyl esters of lauric acid, lauryl trimethyl ammonium chloride, decyl amine acetate, lauryl dimethyl ethyl ammonium chloride, the lactic acid and citric acid and other acid salts of stearyl-1-amidoimidazoline with methyl chloride, benzyl chloride, chloroacetic acid, their salts, and the like.

In some embodiments, the compositions of the present invention may use an anionic surfactant in combination with a polyol compound and/or a cross-linked polyacrylate polymer used as an excipient in pharmaceutical compositions. Either or both of a polyol compound or a cross-linked polyacrylate polymer may be included (i.e., a polyol compound and/or a cross-linked polyacrylate polymer may be included in the composition).

Polyol compounds include, but are not limited to ethylene glycol, propylene glycol, butylene glycol, hexylene glycol, propylene glycol monocaprylate and the like.

In some embodiments, the polyol compound is a compound that may exist in the form of one or more stereoisomers, wherein one or more of those stereoisomers is therapeutically active. In some embodiments, the polyol compound comprises a therapeutically active stereoisomer that is substantially free of other stereoisomers. In other embodiments, the polyol compound comprises a therapeutically active stereoisomer that has less than about 95%, less than about 70%, less than about 40%, less than about 20%, less than about 10%, less than about 5%, less than about 1%, less than about 0.5%, or less than about 0.1% by weight of other stereoisomers.

Cross-linked polyacrylate polymers include, but are not limited to, one or more of polyacrylates, carbomers (e.g., Carboxypolymethylene), polyvinyl acetates, polymethylmethacrylates, hyaluronic acids, polycarboxylated polysaccharides (e.g. alginic acid), carboxyalkyl-polysaccharides (e.g. carboxymethyl starch, carboxyethyl-cellulose), celluloses, dextrans, and the like.

In contrast to current therapies, the present invention advantageously provides for compositions and methods that have minor or no side effects, takes action through a safe local/topical methodology, with minimal recurrence of the manifestations of the viral skin infections; the side effects and recurrence of which are very common with current therapies.

One of the added advantages of the invention is that it can also be applied topically to ensure local action and eliminates the potential side effects associated with oral or systemic administration. In one embodiment, the local/topical application of the compositions of the invention and its therapeutic effects are made possible by the presence of an anionic or amphoteric surfactant in combination with at least one polyol compound and/or a cross-linked polyacrylate polymer, which create the viral inhibitory effects and consequential anti-viral effects of the compositions of the invention.

Example Compositions for Topical Administration

Having described the invention with reference to certain embodiments, other embodiments will become apparent to one skilled in the art from consideration of the specification. The invention is further defined by reference to the following examples. It will be apparent to those skilled in the art that many modifications, both to materials and methods, may be practiced without departing from the scope of the invention.

Suitable pharmaceutically acceptable excipients for the topical compositions of the invention may be any topically acceptable effective excipient known by those skilled in the art. Suitable excipients include, but are not limited to, emulsifying agents, stiffening agents, rheology modifiers or thickeners, emollients, preservatives, humectants, alkalizing or buffering agents, solvents, other surfactants and polyols than the ones described for the treatment of viral skin infections

Suitable emulsifying agents include, but are not limited to, cetyl alcohol, cetostearyl alcohol, stearyl alcohol, carboxypolymethylene, polycarbophil, polyethylene glycol, and sorbitan esters. Suitable stiffening agents include, but are not limited to, stearyl alcohol, cetostearyl alcohol, and cetyl alcohol. Suitable rheology modifiers or thickeners include, but are not limited to, carbomers such as Carbopol®, and polyoxyethylene tallow amines such as Ethomeen®. Suitable emollients include, but are not limited to, white petrolatum (while vaseline), liquid petrolatum (liquid vaseline), paraffin, or aquaphor. Suitable preservatives include, but are not limited to, antimicrobial preservatives such as nipagin (methyl hydroxybenzoate), nipasol (hydroxybenzoate), butylparaben, ethylparaben, methylparaben, propyl paraben 10 potassium, and propyl paraben sodium. Suitable alkalizing or buffering agents include, but are not limited to, sodium hydroxide and potassium hydroxide. Suitable solvents include, but are not limited to, water.

The topical compositions of the invention may be in the form of a gel, cream, ointment, liquid, suspension, solution, emulsion, foam, patch, aerosol or the like for topical administration. Typically, the composition is administered by spreading (e.g., a gel, cream, or ointment) or spraying (e.g., a liquid) onto the affected area of the skin. In one embodiment, the composition may be provided in a kit for the treatment of the conditions and manifestations of the skin infection, wherein the kit includes a unit dose of a surfactant or composition in accordance with embodiments of the present invention described herein. The kit may further include a label or printed instructions for the administration of the composition to treat the manifestations and conditions associated with the skin infections.

In one embodiment, the composition is in the form of a cream. Typically, the cream compositions of the present invention comprise an anionic or amphoteric surfactant and a polyol compound with one or more of an emulsifying agent, a stiffening agent, another surfactant, an emollient, a preservative, an alkalizing or buffering agent, an antioxidant and a solvent. Table 1 below provides the contents of an example composition in the form of a cream.

TABLE 1
Cream Composition Example

| Illustrative Cream Composition | Amount |
|---|---|
| Ingredients | (% by weight of the composition) |
| Stiffening agent | about 1%-45% |
| Cross-linked polyacrylate polymers | about 0.1%-50% |
| Anionic or amphoteric surfactant | about 0.1%-50% |
| Preservative | about 0.01%-0.6% |
| Polyol compound | about 0.1%-50% |
| Alkalizing or buffering agent | about 0.01%-3% |
| Antioxidant | about 1%-15% |
| Emollient | about 1%-50% |
| Solvent (e.g., distilled water) | q.s. (e.g., 20%-80%) |

In one embodiment, the composition is in the form of gel. Typically, the gel compositions of the invention comprise an anionic or amphoteric surfactant and one or more of a rheology modifier or thickener, an alkalizing or buffering agent, and a solvent. Table 2 below provides the contents of an example composition in the form of a gel.

TABLE 2
Gel Composition Example

| Illustrative Gel Composition | Amount |
|---|---|
| Ingredients | (% by weight of the composition) |
| Anionic or amphoteric surfactant | about 0.1%-50% |
| Polyol compound | about 0.1%-50% |
| Cross-linked polyacrylate polymer | about 0.1%-50% |
| Rheology modifier or thickener | about 0.5%-2% |
| Alkalizing or buffering agent | about 0.5%-10% |
| Solvent (e.g., distilled water) | q.s. (e.g., 20%-80%) |

Typically, the composition comprising the anionic or amphoteric surfactant in combination with a polyol compound and/or a cross-linked polyacrylate polymer are administered to the subject in a total daily dose of up to about 500 mg/cm² of skin. The total daily dose may be delivered once per day, or divided between multiple doses. In some embodiments, the composition of the invention may be administered 1, 2, or 3 times per day. In other embodiments, the composition may be administered onto an affected skin area 3 to 14 times weekly by spreading or spraying of the composition onto the affected skin area.

Female Patient Study for HPV Examples

The new methods and topical compositions for treatment of viral skin infections and their manifestations post infection are based on a randomized double-blind clinical trial in patients with manifestations of anogenital warts (caused by HPV) determined by visual assessment and PCR assays. The results demonstrated that administration of the formulation comprising the anionic or amphoteric surfactant, in this patient population, was safe and had minimal side effects, such as skin irritations and lesions, which are potential and concerning side effects of existing therapies, and which create major patient compliance issues.

A completed trial is projected to include 100 patients, 50 of whom are projected to be administered the topical formulation of the present invention. So far 82 patients have been enrolled in the study as of the filing date of the present patent application. 38 patients have administered the topical formulation in accordance with embodiments of the present invention, and administration was repeated as necessary. 100 outpatients will be recruited at the end of the study, although 38 have already administered the formulation (a total of 50 patients are anticipated to administer the topical preparation) comprising an anionic surfactant in combination with at least one polyol compound and/or a cross-linked polyacrylate polymer for self administration, in a double-blind randomized manner. All of the participants were female subjects 18 years and older, all of whom were diagnosed with anogenital condylomata acuminata (HPV).

Criteria for inclusion in the study included the following:
≥18 years;
In good general health;
Diagnosed with genital condylomata acuminata using DNA typing with a type specific, sensitive and quantitative human papilloma virus (HPV) polymerase chain reaction (PCR) assay in the biopsy obtained by cervical smear;
Negative pregnancy test (for women who are able to become pregnant);
Subject is in general good health based on medical history and physical examination;
Subject is able to communicate effectively with study personnel;
Subject is considered reliable, willing, and cooperative in terms of compliance with the protocol requirements; and
Subject voluntarily gives written informed consent to participate in the study.

Criteria for exclusion from the study included the following:
Women who are pregnant, lactating or planning to become pregnant during the study;
Subject has current or a history of cancer of the cervix;
Subject has clinically significant (CS) gynecological abnormalities that could interfere with study evaluation, in the judgment of the investigator (e.g. prolapse, myoma, fibroid, hysterectomy);
Subject has a history of malignant cancer, except the following adequately treated cancers: basal cell carcinoma, or dermatological squamous cell carcinoma;
Subject was administered with another investigational drug or vaccine for the treatment of warts within 30 days prior to the screening visit or is participating in any other study;
Subject has a known hypersensitivity to naltrexone or any of the excipients;
Subject has a history of severe reaction to any drug;
Any inflammatory conditions of the skin or unhealed skin after any previous drug or surgical treatment in the treatment area;
Any organ transplant patients;
Evidence of clinically significant or unstable disease (such as stroke, heart attack);
Have any of the following conditions: HIV infection; outbreak of herpes genitalia in the wart areas; dermatological disease (e.g., psoriasis) or skin condition in the wart areas;
Have received specific treatments in the treatment area(s) within the designated time period prior to study treatment initiation;
Alcohol abuse i.e. regular use of more than 2 units of alcohol per day or a history of alcoholism or drug/chemical abuse (one unit of alcohol equals ½ l of beer, 200 ml wine or 50 ml of spirits) or recovered alcoholics; and
Drug abuse; positive drug screen or history of drug abuse (amphetamines, methamphetamines, cannabinoids, benzodiazepines, cocaine, opioids, barbiturates).

The diagnosis was made using DNA typing with a type specific, sensitive and quantitative human papilloma virus (HPV) polymerase chain reaction (PCR) assay in the biopsy obtained by cervical smear. The DNA typing for HPV was done using F-HPV Typing™ by Molgentix, a fluorescent PCR test kit with automated genotyping for HPV types 6, 11, 16, 18, 31, 33, 35, 39, 45, 51, 52, 56, 58, 59, 68. F-HPV Typing™ is intended to be used to amplify DNA extracted from samples such as biopsies or anogenital swabs.

After the baseline evaluation, patients returned for office visits at weeks 2 (second visit) and 4 (third visit). A visual assessment for possible side effects and wart count was done at each visit. If treatment was successful in the earlier visits, the treatment with the topical composition containing the anionic surfactant was halted but the patient still had to complete the remainder of her visits to check for the recurrence of HPV. If the investigator decided to prolong the treatment, the subjects were evaluated once more at week 6 (fourth visit) and at the end of the treatment deadline, which was 12 weeks (fifth and final visit).

Safety variables included Adverse Reactions (AER), Local Skin Reactions (LSRs), and number of patients who took rest periods during the treatment period. If present, local skin reactions in the treatment and/or immediate surrounding area were assessed as: erythema, edema, weeping/exudate, flaking/scaling/dryness, and/or erosion/ulceration. LSRs were visually assessed by investigator at each visit.

Cumulative Results of Female Patient Study for HPV

Out of the 82 patients enrolled in the study, 38 were given a topical formulation containing an anionic surfactant, in combination with at least one polyol compound and/or a cross-linked polyacrylate polymer in accordance with embodiments of the present invention.

During the study patients were assessed and questioned by the investigator to determine the efficacy and possible side effects of the formulation. 32 patients out of 38 patients had complete clearance using the surfactant composition of the present invention, 2 patients did not have complete clearance, and 4 patients dropped out of the study for reasons unrelated to the topical formulation. Thus, 32 out of 34 patients that adhered to the use of the formulation had complete clearance for a greater than 90% complete clearance rate. More than 50% of the patients using the formulation that can be devised according to the present invention, had complete clearance of genital warts within the first 2 weeks. 20% of patients had complete clearance within the first 4 weeks, and another 20% had complete clearance within the first 6 weeks.

The study has provided evidence of unparalleled clinical effect of the topical composition of the present invention. The study led to the discovery that the topical administration of an anionic or amphoteric surfactant in combination with at least one polyol compound and/or a cross-linked polyacrylate polymer had a positive effect on more than 90% of the study participants and treated the conditions and manifestations associated with genital HPV with minimal side effects. These viral infections and their manifestations are not adequately treated with existing drugs including interferons, immiquimod, and DNA synthesis inhibitors such as nucleoside, purine and prymidine analogues.

By varying the amount of the composition or dosage, the topical administration of the anionic or amphoteric surfactant in combination with at least one polyol compound and/or a cross-linked polyacrylate polymer can be further optimized based on the present disclosure to elicit a maximal improvement in the treatment of skin conditions such as those caused by HPV, HSV-1, HSV-2, and/or herpes zoster.

The results of the study are significant and unparalleled because nearly all of the drugs that have been previously used to treat HPV had both systemic and local side effects and did not have as high a success rate as with the treatment using the present invention. In particular, the HPV strains described above have been one of the most difficult strains to treat, and until the present invention, prior treatments have only had a 52% complete clearance rate of warts with treatment periods going up to 16 weeks using imiquimod cream 5%.

In comparison, the study with the topical formulation of the present invention has demonstrated a complete clearance rate with more than 90% of patients, without recurrence in a maximum time period of 6 weeks, with most of the patients being completely manifestation-free within the first 2 weeks. This is unprecedented in treating the manifestations of HPV, especially considering that only minimal side effects, such as a minor burning sensation occurred and these were not significant enough for the patients to drop out of the study.

Individual Female Patient Examples:

Patient #4 was a 42-year-old female diagnosed with a HPV infection as manifested by warts in the genital region, with close proximity to the vulva. She was positive for HPV type 16. Her anogenital region was clear of warts at her second visit after the daily topical administration of the anionic surfactant formulation. She did not report any side effects associated with the treatment and recurrences were not observed during the follow-up visits. The treatment was not prolonged and the patient was clear of warts and conditions or manifestations associated therewith at the end of the 12 week period.

Patient #16 was a 44-year-old old female diagnosed with a HPV infection as manifested by warts in the genital region. She was positive for HPV type 16. Her anogenital region was clear of warts at her second visit, after the daily topical administration of the anionic surfactant formulation. She did not report any side effects associated with the treatment and recurrences were not observed during the follow-up visits. The treatment was not prolonged and the patient was clear of warts and conditions or manifestations associated therewith at the end of the 12 week period.

Patient #23 was a 31-year-old female diagnosed with a HPV infection as manifested by warts in the genital region. She was positive for HPV type 11-16-68. Her anogenital region was clear of warts at her second visit, after the daily topical administration of the anionic surfactant formulation. She did not report any side effects associated with the treatment and recurrences were not observed during the follow-up visits. The treatment was not prolonged and the patient was clear of warts and conditions or manifestations associated therewith at the end of the 12 week period.

Patient #32 was a 48-year-old female diagnosed with a HPV infection as manifested by warts in the genital region. She was diagnosed with atypical squamous cells of undetermined significance (ASC-US) and turned out to be positive for HPV type 31. Her anogenital region was clear of warts at her second visit, after the daily topical administration of the anionic surfactant formulation. She did not report any side effects associated with the treatment and recurrences were not observed during the follow-up visits. The treatment was not prolonged and the patient was clear of warts and conditions or manifestations associated therewith at the end of the 12 week period.

Patient #55 was a 27-year-old female diagnosed with a HPV infection as manifested by warts in the genital region. She was positive for HPV type 6-68. Her anogenital region was clear of warts at her second visit, after the daily topical administration of the anionic surfactant formulation. She experienced minor itching and burning associated with the treatment, and recurrences were not observed during the follow-up visits. The treatment was not prolonged and the patient was clear of warts and conditions or manifestations associated with HPV therewith as of her last visit.

Additional Patient Examples:

Compounds of the present invention were given for self-administration as a cream formulation to four HSV-2 patients with genital lesions (3 male, 1 female). Two weeks (+/−2 days) after daily topical administration of the formulation in accordance with embodiments of the present invention disclosed herein, the lesions were completely cleared in all four patients. One patient had a minor burning sensation which was not significant enough for the patient to halt the administration.

Individual Male Patient Examples:

Male Patient #1 was a patient with a genital lesion having a surface area of approximately 1 cm² (calculated by multiplying the length and width of the lesion) and diagnosed with HSV-2. After 13 days of daily topical administration on the lesion with an embodiment of the formulation of the present invention as disclosed herein, the lesion was completely cleared and there were no side effects.

Male Patient #2 was given a cream formulation in accordance with embodiments of the present invention disclosed herein for self-administration. The male patient had reactivation of the VZV, namely, the herpes zoster disease. The patient was in the acute eruptive phase and the skin infection had manifested itself in the form of vesicules on the skin, mainly on the upper right dorsal region. The patient had a history of recurring herpes zoster eruptions, and the infection was diagnosed as herpes zoster. 7 days after daily topical administration of the formulation in accordance with embodiments of the present invention disclosed herein, the lesions were completely cleared and surprisingly the patient had less neuropathic pain compared with his previous experiences with the herpes zoster disease.

The foregoing embodiments and examples have been provided merely for the purpose of explanation and are in no way to be construed as limiting of the present invention. While the present invention has been disclosed with reference to embodiments, the words used herein are intended to be words of description and illustration, rather than words of limitation. While the present invention has been described with reference to particular materials and embodiments, the present invention is not intended to be limited to the particulars disclosed herein. Rather, the present invention extends to all functionally equivalent structures, materials, and uses, such as are within the scope of the appended claims. Changes may be made, within the purview of the appended claims, as presently stated and as may be amended, without departing from the scope and spirit of the present invention. All terms used in this disclosure should be interpreted in the broadest possible manner consistent with the context.

1-25. (canceled)

26. A method of treating a post infection skin condition caused by a double stranded DNA virus from a Papillomaviridae family, the method comprising administering to an individual in need of such treatment a composition comprising an anionic or amphoteric surfactant.

27. The method of claim 26, wherein the anionic surfactant is selected from the group consisting of sodium lauryl sulphate, sodium dodecyl sulphate, potassium dodecyl sulphonate, sodium dodecyl benzene sulphonate, sodium salt of lauryl polyoxyethylene sulphate, lauryl polyethylene oxide sulfonate, dioctyl ester of sodium sulphosuccinic acid or sodium lauryl sulphonate, ammonium lauryl sulfate, sodium 2-ethylhexyl sulfate, sodium octyl sulfate, lithium lauryl sulfate and a pharmaceutically acceptable salt thereof, and a combination thereof.

28. The method of claim 26, wherein the skin condition includes one or more of common warts, plantar warts, flat warts, anogenital warts, anal dysplasia, epidermodysplasia verruciformis, focal epithelial hyperplasia, oral papillomas and laryngeal papillomatosis.

29. The method of claim 26, wherein the double stranded DNA virus from the Papillomaviridae family includes the human papilloma virus.

30. The method of claim 26, wherein the composition further comprises a hydrophilic moiety selected from the group consisting of a carbonate, a sulfonate, and a sulfate.

31. The method of claim 26, wherein the composition further comprises a polyol compound and/or a cross-linked polyacrylate polymer.

32. The method of claim 31, wherein the polyol compound is selected from the group consisting of ethylene glycol, propylene glycol, butylene glycol, hexylene glycol, propylene glycol monocaprylate, and a combination thereof.

33. The method of claim 31, wherein the cross-linked polyacrylate polymer is selected from the group consisting of polyacrylates, carbomers, polyvinyl acetates, polymethylmethacrylates, hyaluronic acids, polycarboxylated polysaccharides, carboxyalkyl-polysaccharides, celluloses, dextrans, and a combination thereof.

34. The method of claim 26, wherein the surfactant is between 0.1% to 20% by weight of the composition.

35. The method of claim 26, wherein the step of administering comprises topically administering.

36. The method of claim 26, wherein the composition is in the form of a gel, a cream, an ointment, a liquid, a suspension, a solution, an emulsion, a foam, a patch, or an aerosol.

37. The method of claim 26, wherein composition is administered onto affected skin area 1 to 3 times daily by spreading or spraying.

38. The method of claim 26, wherein the composition is administered onto affected skin area 3 to 14 times weekly by spreading or spraying.
